(19) United States
(12) Patent Application Publication
(10) Pub. No.: US 2015/0141764 A1
(43) Pub. Date: May 21, 2015

(54) DISTRIBUTED SENSING DEVICE FOR REFERENCING OF PHYSIOLOGICAL FEATURES

(71) Applicant: KONINKLIJKE PHILIPS N.V., EINDHOVEN (NL)

(72) Inventors: Godefridus Antonius Harks, Rijen (NL); Martinus Bernardus Van Der Mark, Best (NL); Gert Wim 'T Hooft, Eindhoven (NL)

(21) Appl. No.: 14/401,554

(22) PCT Filed: May 28, 2013
(86) PCT No.: PCT/IB2013/054395

§ 371 Date: Nov. 17, 2014

Related U.S. Application Data
(60) Provisional application No. 61/657,081, filed on Jun. 8, 2012.

(51) Int. Cl. A61B 5/00; A61B 5/026; A61B 5/0215; A61B 5/0205; A61B 5/1455

(52) U.S. Cl. CPC A61B 5/0084; A61B 5/0205; A61B 5/14552; A61B 5/02154; A61B 5/0261; A61B 5/02055
USPC 600/301; 600/339; 600/342; 600/478

(57) ABSTRACT

A distributed sensor and a method for identifying an internal anatomical landmark (R) includes inserting (502) a distributed sensing device (212) into a volume of a body and extending (504) a portion of a length of the distributed sensing device beyond an area of interest. Parameters are measured (506) using sensors (202) located along the length of the distributed sensing device (212), and a transition region is determined (510) based upon a parameter value difference between adjacent sensors. A location of an anatomical landmark is assigned (512) using the transition region.

This disclosure relates to medical devices and more particularly to shape sensing optical fibers in medical applications for locating a physical reference feature from which other measurements can be made.

In minimally invasive therapies, image guidance is needed to navigate interventional tools such as catheters, needles and deployment devices to a correct location in the body and to ensure that the therapy is applied to a correct area of tissue. Devices can be visualized using imaging modalities such as X-ray, magnetic resonance (MR) and ultrasound (passive modes). On the other hand, the devices can be functionalized with specific sensors such that they can be tracked (active modes).

Current localization technologies determine the location of the device in 3D space with respect to a reference (e.g., patches), which is typically outside the body. However, for a treatment procedure, an operator would prefer information on the location of the device with respect to the true anatomy, which may be moving.

External tracking mechanisms to monitor the location of these devices inside the body can be used as an adjunct to guide navigation during interventional procedures. Many tracking technologies exist, and each has its own advantages and disadvantages. For example, electromagnetic tracking systems are able to localize a tip of a device while it is embedded inside the body. However, metal in the medical environment may disturb the electromagnetic field and reduce the accuracy of the measurement. Another example may include an impedance based tracking system that localizes a device inside the body by measuring the electrical potential across body tissue. A large degree of tissue heterogeneity in the body challenges the accuracy of this method.

An ultrasound-based tracking system uses pulsed ultrasound to triangulate the location of the device. This system requires a fluid environment without abrupt changes in acoustic impedance or material density so that assumptions about the speed of sound and acoustic wave propagation are accurate. For example in the lungs, the tissue/air boundaries may present a problem. Similarly, bone/tissue boundaries are problematic. Optical tracking systems rely on line-of-sight to the tracked device, which greatly limits the applicability of this technology to rigid instruments that are partially outside the body. In conventional tracking systems, only a single point or a small number of points close to a tip of a catheter are usually tracked.

Anatomical structures can be visualized using imaging systems (imaging). Alternatively, anatomical structures can be reconstructed by using a catheter that is provided with a tracking sensor and moved along an anatomical structure (reconstruction). Imaging 3D anatomy may include employing 3D anatomical information of a target anatomical structure obtained from pre-recorded images (e.g., computed tomography (CT), MR, etc.) or rotational angiography after contrast injection. Alternatively, 3D ultrasound (e.g., TEE (Trans-Esophageal Echo), ICE (Intra Cardiac Echo), etc. can be used to visualize the 3D anatomy. Reconstruction of 3D structures using tracking devices may be employed. In cardiac electrophysiology procedures, tracking technologies are often used for electroanatomic mapping to reconstruct the 3D anatomy of the heart and in particular the left atrium for the treatment of atrial fibrillation. Several mapping technologies exist and are helpful in determining the location of a catheter with respect to tissue anatomy. (Electro-anatomical) mapping systems only show indirect representations and not the true anatomy. The accuracy of such systems is limited to ˜1-2 mm.

In accordance with the present principles, a method for identifying an internal anatomical landmark includes inserting a distributed sensing device into a volume of a body and extending a portion of a length of the distributed sensing device beyond an area of interest. Parameters are measured using sensors located along the length of the distributed sensing device, and a transition region is determined based upon a parameter value difference between adjacent sensors. A location of an anatomical landmark is assigned using the transition region.

Another method for identifying an internal anatomical landmark, in accordance with the present principles, includes inserting a distributed fiber optic sensing device into a volume of a body; extending at least a portion of a length of the distributed sensing device beyond an area of interest such that the length of the distributed sensing device includes sensors on different sides of the point of interest; measuring one or more parameters from surrounding tissue using the sensors located along the length of the distributed sensing device; determining a transition region where a gradient point occurs between the sensors to associate the gradient point with one or more positions of the sensors along the length; and assigning a location of an anatomical landmark to a sensor nearest to the gradient point.

A system for identifying an internal anatomical landmark includes a processor and a memory coupled to the processor. A distributed sensing device is insertable in a volume of a body and includes a plurality of sensors distributed over a length of the sensing device. A sensing and interpretation module is stored in the memory and is configured to measure distributed sensing data collected from the sensors over a length of the distributed sensing device such that when the distributed sensing device is deployed in the body a gradient in the distributed sensing data is determined over one or more measured parameters to identify an anatomical landmark as a reference position for the distributed sensing data.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

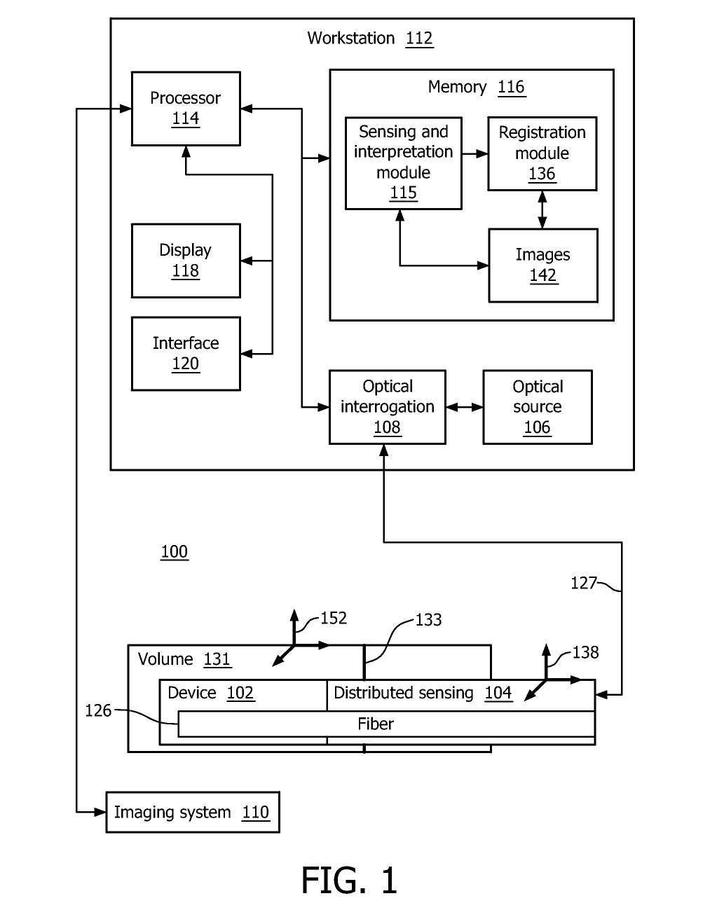

FIG. 1 is a block/flow diagram showing a system for distributed sensing which is employed for determining an internal anatomical landmark in accordance with one embodiment;

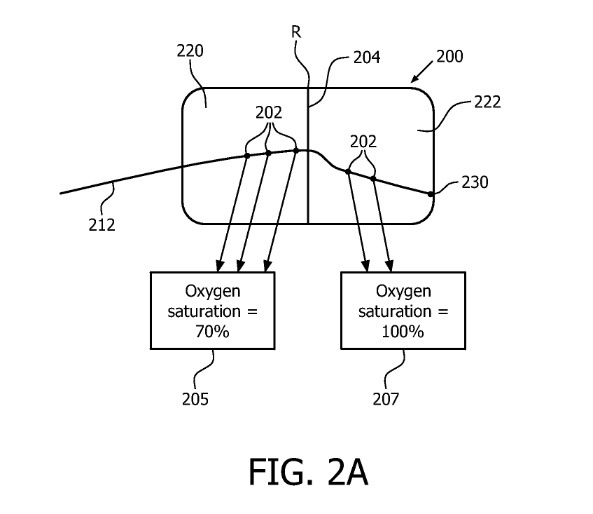

FIG. 2A is a schematic diagram showing a distributed sensing device disposed between atria of a heart and corresponding oxygen saturation in accordance with one illustrative embodiment;

FIG. 2B is a schematic diagram showing a distributed sensing device disposed between atria of a heart and corresponding pressure in accordance with one illustrative embodiment;

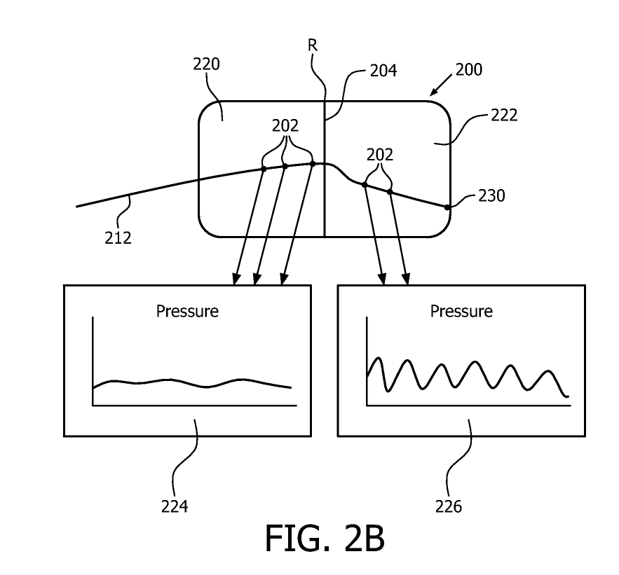

FIG. 3 shows diagrams depicting different sensor configurations for the distributed sensing device in accordance with illustrative embodiments;

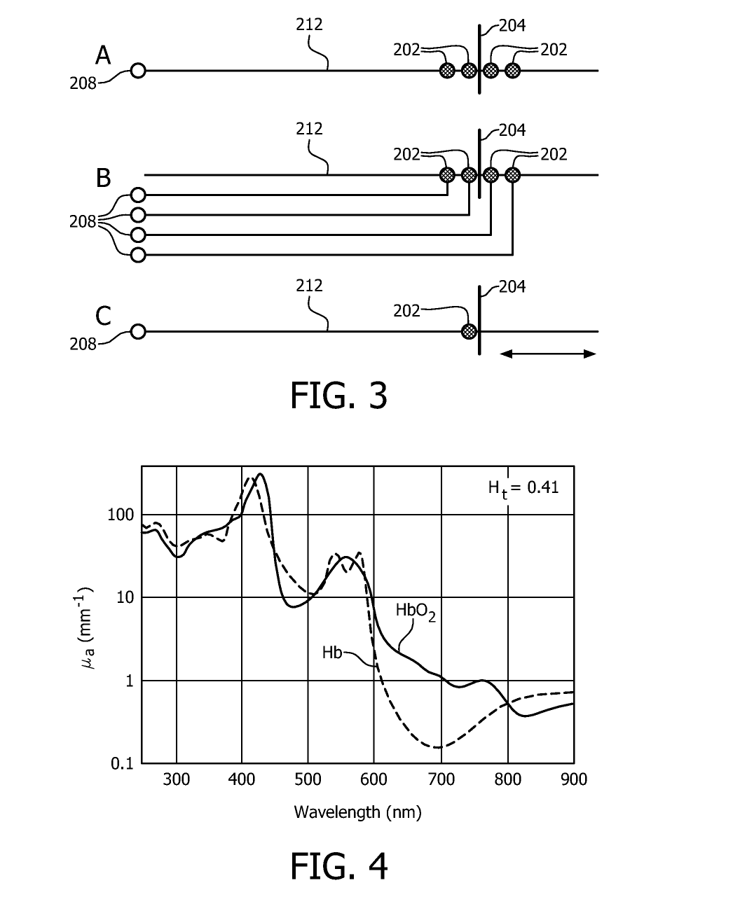

FIG. 4 is a plot depicting absorption spectra showing spectral differences between hemoglobin and oxygenated hemoglobin in accordance with illustrative embodiments;

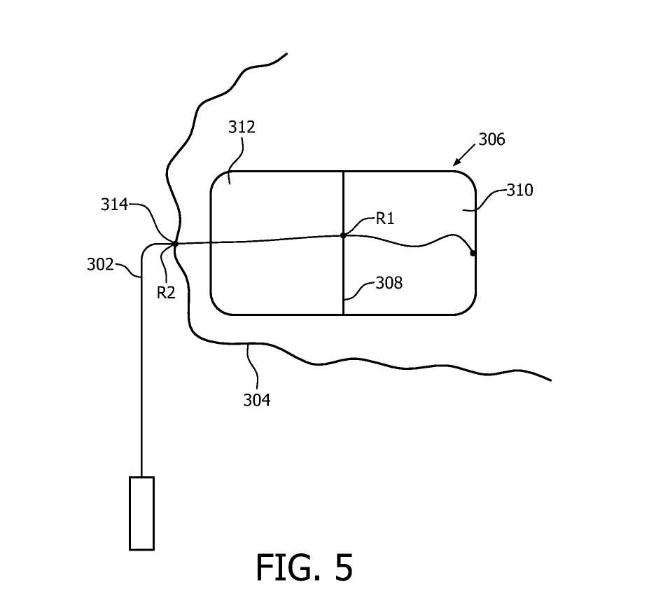

FIG. 5 is a schematic diagram showing a distributed sensing device with two reference points defined to provide dynamic references in accordance with one illustrative embodiment;

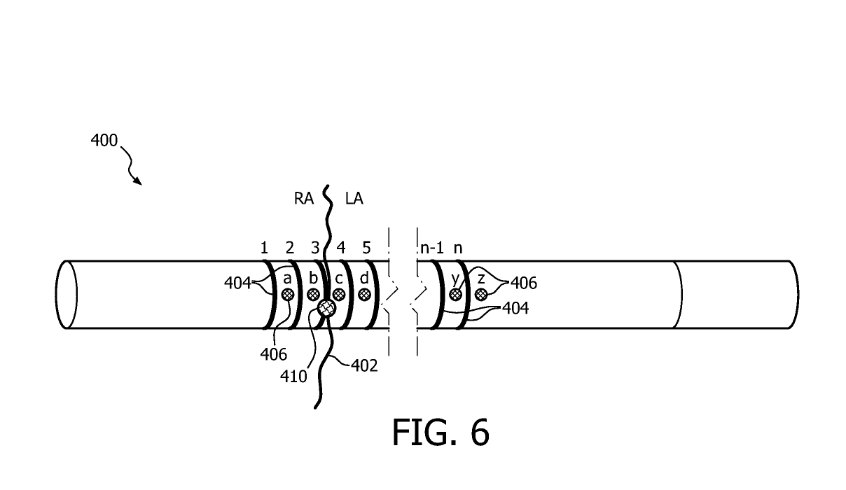

FIG. 6 is a diagram showing a distributed sensing device with demarcations for rendering the device visible in images in accordance with one illustrative embodiment; and

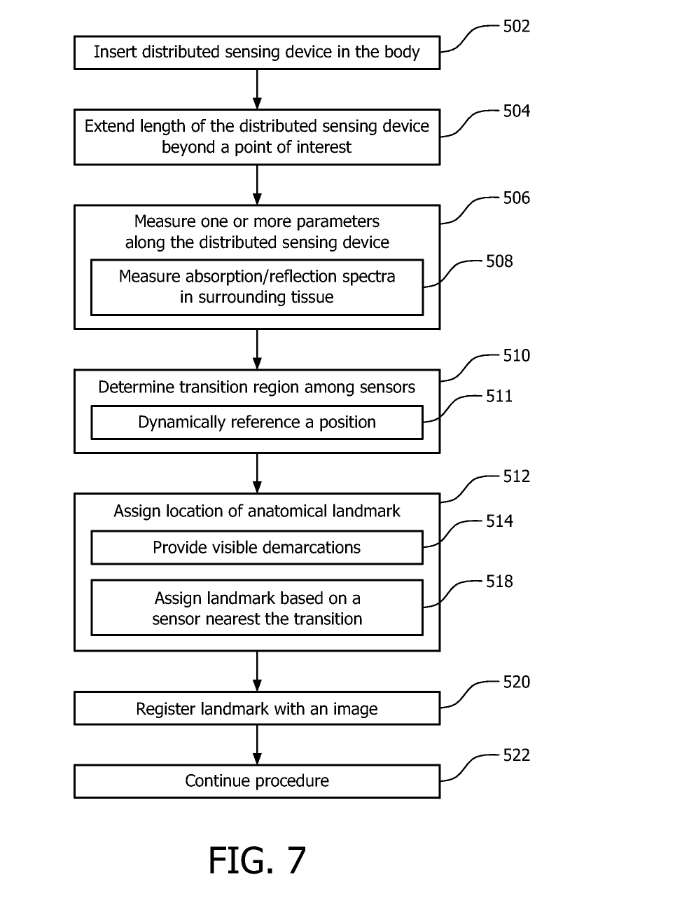

FIG. 7 is a block/flow diagram showing a method for distributed sensing which is employed for determining an internal anatomical landmark in accordance with another embodiment.

In accordance with the present principles, systems and methods are provided that employ Fiber Optic Shape Sensing and Localization (FOSSL) technology to locate and track an internal anatomical feature. FOSSL technology or optical fiber shape sensing makes optical fibers sensitive to strain and temperature. Surrogate variables such as flow, inflammation, tissue pressure/swelling, tissue contact, etc., can be measured indirectly (using, in the case of flow, for example, temperature gradients of indicator dilution). The fibers, when embedded in a vessel, can provide the 3D shape and dynamics of the vasculature, as well as flow information to help detect anatomical features within the body.

In one embodiment, a procedure is performed using an intraluminally disposed shape sensing fiber optic device inserted into a vessel or organ, e.g., a chamber of the heart. A three-dimensional (3D) reconstruction of shape and flow information of the vessel or organ (as obtained from shape sensing fiber) is provided, which permits computations for locating a reference feature or anatomical landmark. Registration between a shape sensing coordinate frame and the reference feature can be made. Anatomical landmarks can be detected by employing transitions in physiological parameters. Physiological parameters may include, e.g., oxygen saturation, CO₂ saturation, pressure, temperature, pH, flow rate, etc. These parameters may include values that demonstrate a gradient, preferably a steep gradient over an anatomical landmark (e.g., trans-septal, coronary ostium, valve plane, etc.) or at a diseased area (e.g., aneurysm, stenosis, tumor margin, etc.).

The present embodiments employ spatially distributed sensing along an elongated device to assess the exact location of an anatomical landmark such that the position of the device can be determined with respect to the true anatomy. Physiological parameters that can be measured comprise oxygen saturation, CO₂ concentration, pH, pressure, flow and temperature. For example, blood oxygenation can be measured with optical fiber sensors and the exact location of the atrial septum can be determined based on transseptal difference in oxygen saturation.

Current localization technologies use an external reference to determine the 3D coordinates of a device in a 3D space, which limits the accuracy of the position with respect to the targeted anatomy. To improve the accuracy, knowing the exact location of a device internally with respect to the targeted anatomical structure would be advantageous. By determining the position of an anatomical landmark such as the septum with a device based on distributed sensing, a dynamic reference point (e.g. anatomical landmark) can be assigned. This reference can be used to align (pre-recorded) anatomy data with device tracking and enhance the accuracy of localization and mapping and would be particularly beneficial for a moving structure like the heart, for example.

It should be understood that the present invention will be described in terms of medical devices for performing therapy and, in particular, minimally invasive therapy or other procedures; however, the teachings of the present invention are much broader and are applicable to any internal procedure. In some embodiments, the present principles are employed in tracking or analyzing complex biological or mechanical systems. In particular, the present principles are applicable to internal tracking procedures of biological systems, procedures in all areas of the body such as the lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk read only memory (CD-ROM), compact disk readwrite (CD-RW), Blu-Ray™ and DVD.

Referring now to the drawings in which like numerals represent the same or similar elements and initially to FIG. 1, a system 100 for monitoring a lumen, such as a blood vessel using shape sensing enabled devices is illustratively shown in accordance with one embodiment. System 100 may include a workstation or console 112 from which a procedure is supervised and/or managed. Workstation 112 preferably includes one or more processors 114 and memory 116 for storing programs and applications. Memory 116 may store a sensing and interpretation module 115 configured to interpret feedback signals, preferably optical signals, from a distributed sensing device or system 104. The distributed sensing device 104 may include fiber optic shape sensing and localization, which measures a whole size and shape of the device 104, yielding a true 3-dimensional curve of, for example, a catheter, guide wire or other device with which the fiber optic shape sensing device is employed.

Optical sensing module 115 is configured to use the optical signal feedback (and any other feedback, e.g., electromagnetic (EM) tracking) to reconstruct deformations, deflections and other changes associated with a medical device or instrument 102 and/or its surrounding region. Sensing module 115 may include models and statistical methods for evaluating the shape sensing data to provide geometric relationships and states of the sensing device or system 104. The medical device 102 may include a catheter, a guidewire, a probe, an endoscope, a robot, an electrode, a filter device, a balloon device, or other medical component, etc.

The sensing device 104 on device 102 may include one or more optical fibers 126 which are coupled to the device 102 in a set pattern or patterns. The sensing device 104 connects with an optical interrogator 108 that provides selected signals and receives optical responses. The optical fibers receive and reflect optical signals using the optical interrogation system 108, which includes or is coupled to a light source 106. The optical source 106 may be provided as part of the interrogator 108 or as a separate unit for providing light signals to the sensing device 104. The optical fibers 126 connect to the workstation 112 through cabling 127. The cabling 127 may include fiber optics, electrical connections, other instrumentation, etc., as needed.

Sensing system 104 with fiber optics may be based on fiber optic Bragg grating sensors. A fiber optic Bragg grating (FBG) is a short segment of optical fiber that reflects particular wavelengths of light and transmits all others. This is achieved by adding a periodic variation of the refractive index in the fiber core, which generates a wavelength-specific dielectric mirror. A fiber Bragg grating can therefore be used as an inline optical filter to block certain wavelengths, or as a wavelength-specific reflector.

A fundamental principle behind the operation of a fiber optic Bragg grating is Fresnel reflection at each of the interfaces where the refractive index is changing. For some wavelengths, the reflected light of the various periods is in phase so that constructive interference exists for reflection and, consequently, destructive interference for transmission. The Bragg wavelength is sensitive to strain as well as to temperature. This means that Bragg gratings can be used as sensing elements in fiber optical sensors. In an FBG sensor, the measurand (e.g., strain) causes a shift in the Bragg wavelength.

One advantage of this technique is that various sensor elements can be distributed over the length of a fiber. Incorporating three or more cores with various sensors (gauges) along the length of a fiber that is embedded in a structure permits a three dimensional form of such a structure to be precisely determined, typically with better than 1 mm accuracy. Along the length of the fiber, at various positions, a multitude of FBG sensors can be located (e.g., 3 or more fiber sensing cores). From the strain measurement of each FBG, the curvature of the structure can be inferred at that position. From the multitude of measured positions, the total three-dimensional form is determined.

As an alternative to fiber-optic Bragg gratings, the inherent backscatter in conventional optical fiber can be exploited. One such approach is to use Rayleigh scatter in standard single-mode communications fiber. Rayleigh scatter occurs as a result of random fluctuations of the index of refraction in the fiber core. These random fluctuations can be modeled as a Bragg grating with a random variation of amplitude and phase along the grating length. By using this effect in three or more cores running within a single length of multi-core fiber, the 3D shape and dynamics of the surface of interest can be followed.

The device 102 may be inserted into a volume 131, such as a lumen, e.g., blood vessel or an organ, such as the heart. The optical sensing system 104 is employed as a tracking system such that nodes (e.g., FBG sensors) are employed in a distributed fashion to monitor parameters over a given distance of an anatomy. In this way, the distributed sensors can detect differences in the parameters as a function of distance. The tracking with distributed sensors is employed to find a true anatomical reference 133. This reference 133 is assigned by assessing a position of a gradient in one or more physiological parameters (e.g., pressure, blood oxygenation, temperature, etc.) at a distal portion of the shape sensing device 104, and the reference 133 can move along with the movement of the anatomical structure.

An imaging system 110 may be employed for in-situ imaging of a subject or volume 131 during a procedure. Imaging system 110 may include a fluoroscopy system, a computed tomography (CT) system, an ultrasonic system, etc. The imaging system 110 may be incorporated with the device 102 (e.g., intravenous ultrasound (IVUS), etc.) or may be employed externally to the volume 131. Imaging system 110 may also be employed for collecting and processing pre-operative images to map out a region of interest in the subject to create an image volume for registration with shape sensing space. It should be understood that the data from imaging device 110 may be helpful but is not necessary for performing a mapping in accordance with the present principles. Imaging device 110 may provide a reference position as to where a cavity or other region of interest exists within a body but may not provide all the information that is desired or provide a digitized rendition of the space or be capable of resolving all of the internal features of the space.

Referring to FIGS. 2A and 2B, a transseptal difference between a right atrium 220 and a left atrium 222 in terms of oxygen saturation (FIG. 2A) and pressure profile (FIG. 2B) are illustratively depicted. A distributed sensing device 212 may include a catheter or the like having a fiber optic sensing system, although other medical devices and sensing systems may be employed. The distributed sensing device 212 has a plurality of sensors 202 distributed along its length. In this example, a heart 200 is shown having a left atrium 222 and a right atrium 220 depicted. An atrial transseptal difference in oxygen saturation and blood pressure exists between the two atria 220 and 222 and provides a steep gradient between at least oxygen saturation and blood pressure. Other parameters may be employed as well for determining a gradient difference across and anatomical landmark. For example, in FIG. 2A, the right atrium 220 may have an oxygen saturation of between about 65% to about 80% and in this case is shown as about 70% in block 205. While the left atrium 222 may have an oxygen saturation of between about 97% to about 100% and in this case is shown as about 100% in block 207. In FIG. 2B, notable pressure differences, shown in illustrative plots 224 and 226, occur between the atria 220 and 222 across the septum (transseptal). Using these data, an exact location of an anatomical landmark (e.g., the septum) can be determined based on the steep transitions of the physiological parameters that are related to anatomy. This location can then be used as a dynamic reference point, which can be linked to (pre-recorded) anatomical data and enable accurate positioning and mapping as will be described in greater detail below.

Referring again to FIG. 1, the sensing device 104 collects data related to position in the volume (e.g., blood vessel or organ) 131. This may include the monitoring of motion due to blood flow and temperature fluctuations due to blood flow, etc. The changes or fluctuations caused across a boundary or other physical feature can be monitored and/or accumulated over time to establish the anatomical reference 133. Statistical methods in the sensing module 115 may indirectly compute gradients in the blood vessel or organ 131. The sensing device 104 has its own coordinate system 138, which can be registered to a coordinate system 152 of preoperative or real-time images of the anatomy. These coordinate systems 138 and 152 can be registered so that data feedback from the sensing device 104 can define the anatomical feature or landmark 133.

In one example, a registration method performed by or in conjunction with a registration module 136 may be employed to register the information from the sensing fiber 126 of device 104 onto preoperative or real-time images 142. In this case, the fiber coordinate frame 138 is registered to the coordinate frame 152 of the images. Registration of other images is also contemplated.

During a procedure, the device 102 equipped with the sensing device 104 is inserted into a patient at or near the anatomical landmark 133, such as in a lumen such as a blood vessel or an organ such as a heart. Position and parameter data are collected over a length of the sensing device 104 in a distributed manner. It is preferably to set up the sensing device 104 in a manner that has sensor nodes that straddle a boundary where a gradient in measured parameters exists and can be measured. Dynamic changes are recorded using the sensing device 104. Dynamic changes may be indirectly measured using temperatures differences, blood vessel motion, blood vessel stiffness, oxygen or carbon dioxide saturation, pressure differences, etc.

Workstation 112 includes a display 118 for viewing internal images of the volume 131 (patient) (e.g., real-time images or pre-operative images). Display 118 may permit a user to interact with the workstation 112 and its components and functions, or any other element within the system 100. This is further facilitated by an interface 120 which may include a keyboard, mouse, a joystick, a haptic device, or any other peripheral or control to permit user feedback from and interaction with the workstation 112. The system 100 may include or be employed with other devices and tools as well.

Referring to FIG. 3, a diagram showing three illustrative examples for deploying a distributed sensing device 212, which may include a catheter equipped with an optical fiber sensing system, is depicted in accordance with the present principles. The device 212 may include an optical fiber sensing device having a plurality of sensors 202 distributed along its length. The three different examples for sensing physiological measures at different sites along the device 212 to determine an anatomical landmark will be described in terms of a septum 204 in a heart. The three examples include a case A having multiple sensors 202 connected to a single detector 208 for read-out. Case A needs to employ multiplexing since a single line is employed to carry signals from the sensors 202. Note that the sensors 202 straddle a boundary where the septum 204 is located so that sensors 202 are located on both sides of the boundary.

In a case B, each sensor 202 is connected individually to a separate detector 208. The sensors 202 again straddle the boundary where the septum 204 is located so that sensors 202 are located on both sides of the boundary. In case C, one sensor 202 is connected to one detector 208 and by moving the sensor 202 across the boundary the location of the anatomical landmark (septum 204) can be determined by taking readings over time.

In the cases A, B and C, the sensors 202 measure parameters or sense physiological measures along the elongated device 212 to assess the exact location of the anatomical landmark 204 such that the position of the device 212 can be determined with respect to the true anatomy. The physiological parameters that can be measured may include, e.g., oxygen saturation, CO₂ concentration, pH, pressure, flow, temperature, etc.

In a preferred embodiment, distributed sensing employs optical shape sensing in the device 212 and distributed optical fiber sensors 202 are employed (e.g., FBGs). As another example, distributed detection of blood oxygenation can be performed by applying diffuse reflection spectroscopy on many points along a fiber (e.g., sensor locations) that is integrated in the device 212. Distributed sensing can, for example, be implemented in a single fiber by locally transmitting light through the cladding around the fiber or by employing interferometric methods using different wavelengths (case A). In such cases, light is emitted from the fiber and reflected off surrounding tissues. The changes due to the reflected or absorbed light (e.g., absorption spectra) are detected (by detector(s) 208) to determine parametric differences. For example, optical detection of blood oxygenation is based on the fact that the absorption profile of hemoglobin (Hb) changes upon the binding of oxygen. Upon detection of a transition as an absolute value or using dynamics of a physiological parameter along the device 212, the exact position of an anatomical landmark (septum 204) can be assessed.

Referring to FIG. 4, illustrative absorption spectra of Hb and HbO₂ show a clear difference in absorption distance (μ_{A} in mm⁻¹) versus wavelength (nm). A pronounced difference occurs especially at about 700 nm. This enables physiological and anatomical boundaries to be distinguished and therefore located due to the gradient or difference.

Referring to again to FIGS. 2A and 2B, continuing with the example of the atrial septum 204, an exact location of the atrial septum 204 may be determined by measuring oxygen saturation (FIG. 2A) or pressure gradient (FIG. 2B) at a discrete number of sensors 202 at a distal end portion of a catheter 212. The values measured by the sensors 202 that are positioned in the right atrium 220 are very different from the values measured for the sensors 202 that are positioned in the left atrium 222 (e.g., saturation is 70% in right atrium 220 and ˜100% in left atrium 222). The exact location of the atrial septum 204 can be determined by assessing the location on the catheter 212, equipped with a distributed sensing system, where the measured value for the blood oxygenation shows a steep transition. This specific location on the device is then assigned as a reference R.

In FIG. 2B, pressure differences in the right atrium 220 are very different from the values measured in the left atrium 222, as depicted in blocks 224 and 226, respectively. The exact location of the atrial septum 204 can be determined by assessing the location on the catheter 212, equipped with a distributed sensing system, where the measured value for the pressure shows a steep transition or difference. This specific location on the device is then assigned as the reference R or may be employed to further confirm the results of a measured reference R at a different time or using a different test.

The catheter 212 with distributed sensors 202 at the distal part is employed to measure physiological parameters (oxygen saturation pressure) for assessment of the location of the atrial septum (reference R). Using distributed sensing, the position of reference R is in close proximity to the left atrium 222 as opposed to reference points outside the body that would be employed with conventional systems. The internal reference point R provides a truer point of reference nearer to a point of interest. Once the reference R is determined other points of the device 212 may be determined. For example, a distance for a distal tip 230 of the catheter 212 to the reference R may be determined and may be employed to map out the area relative to the reference R in which the device 212 is disposed.

Determining a reference point R based on distributed sensing has several advantages and may include at least one of the following. The reference R is determined with respect to the anatomical structure of interest (e.g., left atrium), and is therefore more accurate than external references. The location of the reference point R can be updated in real-time and is therefore, insensitive to movement of the anatomical structure (e.g., beating heart) or patient movement. The reference point R as measured by distributed sensing can be used to highlight the location of an anatomical/functional landmark that is not visible or is hardly visible on an imaging modality, e.g., an atrial septum is difficult to see with X-ray imaging when the catheter 212 is in the left atrium. In one example for a heart ablation procedure, a transseptal needle is to be placed in contact with a foramen ovale (septum) for puncturing. After puncture of the foramen ovale, an ablation catheter is guided through the puncture opening and targeted towards pulmonary veins in the left atrium. The location of the atrial septum (foramen ovale) is not normally visible on the X-rays. However, in accordance with the present principles, the location of the atrial septum could be visualized on the image using distributed sensing to define the position and indicate the position in the image.

The reference point R can be used to improve overlay image registration with pre-recorded 3D anatomical data, e.g., an alignment between the ostium or orifice of a coronary artery from pre-recorded 3D data can be made with the reference point R as measured by distributed sensing on a guidewire (i.e., linking pre-recorded 3D information with real-time device position information). A dynamic reference point can be used as input for the 3D reconstruction of a distributed sensing device if at least two locations of an optical shape sensing (OSS) device are known based on anatomical landmarks. For example, FIG. 6 shows an example of dynamic referencing.

Referring to FIG. 5, an optical shape sensing (OSS) device 302 is inserted in a body 304 of a patient and into a heart 306 in this example. The device 302 crosses a septum 308 between a left atrium 310 and a right atrium 312. A reference R1 is determined at the septum 308, as described, to provide a first reference point. A position of the device 302 at an entry point 314, e.g., the groin, into the body 304 can be determined as R2. R2 can be determined from measuring temperature drop from outside the body 304 to the inside of the body 304. Since two points R1 and R2 on the device 302 are known and the shapes of the device 302 are known, an exact 3D orientation of the device 302 can be reconstructed.

Referring to FIG. 6, an illustrative embodiment of another distributed sensing device 400 is shown in accordance with the present principles. Again, the example of determining an internal reference point will employ an atrial septum 402 between left atrium (LA) and right atrium (RA). The location of the atrial septum 402 as detected by distributed sensing device 400 may also be viewed in a fluoroscopy image by employing demarcations 404. The demarcations 404 along the device 400 may include radiopaque material, such as metal or inked contrast dye. The demarcations 404 are preferably spaced between sensors 406. In one embodiment, the demarcations 404 include metal rings formed about the device 400 and can be visualized on an X-ray image.

In the example of FIG. 6, sensors 406 are labeled a-z and demarcations or rings 404 are labeled 1-n. The septum 402 falls on ring number 3 between sensors b and c. Sensors a and b measure oxygen saturation at one level while the remaining sensors c-z measure a second level providing for a noted transition (septum position). Since the metal rings (demarcations 404) are radiopaque, the rings can be seen in the X-ray images and visually indicate the anatomical landmark position and can be employed for image registration with other imaging modalities (e.g., preoperative images). A marker 410 may be placed on the septum position in the X-ray image. Other demarcations 404 may also be employed instead of or in addition to those described. The demarcations 404 may include different shapes, positions, materials, etc. For example, in magnetic resonance imaging (MRI), demarcations 404 other than metal rings can be used for visualizing the location of an anatomical landmark on an MR image, e.g. coils may be employed.

In addition to an assessment of an anatomical landmark, such as e.g., the septum, a coronary ostium, a valve plane, etc., functional landmarks may also be assessed by distributed sensing. The functional landmarks may include, e.g., aneurysms, stenosis, tumor margin, etc. Potential applications for determining anatomical/functional landmarks with distributed sensing may include, e.g., determining a general position of a landmark, image and sensing data registration, visualizing hard to see or invisible landmarks, targeted therapy delivery (e.g., for stent deployment, tumor ablation, etc.), and other applications.

Referring to FIG. 7, a method for identifying an internal anatomical landmark is shown in accordance with illustrative embodiments. In block 502, a distributed sensing device is inserted into a volume of a body. The distributed sensing device may include a fiber optic shape sensing device, and its sensors may include fiber optic sensors disposed along the length of the fiber optic shape sensing device. The volume may include at least one of a lumen or organ of a circulatory system although other anatomical features may also be employed.

In block 504, at least a portion of a length of the distributed sensing device is extended beyond an area of interest (suspected boundary or landmark). In block 506, one or more parameters are measured using sensors located along the length of the distributed sensing device. The parameters may include measuring one or more of oxygen saturation, pressure, flow, pH, carbon dioxide saturation, temperature, etc. In block 508, measuring one or more parameters may include measuring light absorption/reflection spectra of surrounding tissues with a fiber optic shape sensing device.

In block 510, a transition region (e.g., based on gradient or definitive/abrupt changes) is determined based upon at least one parameter value difference between adjacent sensors. In block 511, positions of the distributed sensing device may be dynamically referenced using the location of the anatomical landmark assigned at the transition region and at least one other reference point.

In block 512, a location of an anatomical landmark is assigned using the transition region. The anatomical landmark may include an atrial septum, a coronary ostium, a valve plane or other anatomical feature. The anatomical landmark may include a functional landmark, such as, e.g., an aneurysm, a stenosis, a tumor margin, etc. In block 514, the distributed sensing device may include demarcations visible in an image. A location of an anatomical landmark may be assigned in the image using a demarcation. The images may include real-time images or pre-operative images. In block 518, the location of the anatomical landmark may be assigned based upon a location of a sensor nearest to the transition region. In block 520, the location of the anatomical landmark may be employed to register the anatomical landmark with an image. In block 522, the procedure is continued as needed.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for referencing of physiological features using distributed sensing (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A system for identifying an internal anatomical landmark, comprising:
a processor;
a memory coupled to the processor;
a distributed sensing device insertable in a volume of a body and including a plurality of sensors distributed over a length of the sensing device; and
a sensing and interpretation module stored in the memory and configured to measure distributed sensing data collected from the sensors over a length of the distributed sensing device;
wherein the sensing and interpretation module is configured to determine a gradient over one or more measured parameters in the distributed sensing data collected from sensors located within the body when the distributed sensing device is deployed in the body; and
wherein said sensing and interpretation module is configured to identify an internal anatomical landmark as a reference position for the distributed sensing data based on the gradient in the distributed sensing data.

2. The system as recited in claim 1, wherein the volume includes at least one of a lumen or organ of a circulatory system.

3. The system as recited in claim 2, wherein the one or more parameters includes one or more of oxygen saturation, pressure, flow, carbon dioxide saturation and temperature.

4. The system as recited in claim 1, wherein the anatomical landmark includes one of an atrial septum, a coronary ostium and a valve plane.

5. The system as recited in claim 1, wherein the anatomical landmark includes a functional landmark.

6. The system as recited in claim 5, wherein the functional landmark includes one of an aneurysm, a stenosis and a tumor margin.

7. The system as recited in claim 1, wherein the one or more measured parameters includes light spectra of surrounding tissues with the distributed shape sensing device.

8. The system as recited in claim 1, wherein the distributed sensing device includes demarcations visible in an image.

9. The system as recited in claim 1, with the distributed sensing device includes a distributed fiber optic shape sensing device.

10. A method for identifying an internal anatomical landmark, comprising:
inserting a distributed sensing device into a volume of a body;
extending at least a portion of a length of the distributed sensing device beyond an area of interest;
measuring one or more parameters using sensors within the body located along the length of the distributed sensing device;
determining a transition region based upon at least one parameter value difference between adjacent sensors; and
assigning a location of an anatomical landmark using the transition region.

11. The method as recited in claim 10, wherein the volume includes at least one of a lumen or organ of a circulatory system, and wherein measuring one or more parameters includes measuring one or more of oxygen saturation, pressure, flow, carbon dioxide saturation and temperature.

12. The method as recited in claim 10, wherein the anatomical landmark includes one of an atrial septum, a coronary ostium and a valve plane, wherein the anatomical landmark includes a functional landmark, and wherein the functional landmark includes one of an aneurysm, a stenosis and a tumor margin.

13. The method as recited in claim 10, wherein the distributed sensing device includes a fiber optic shape sensing device and the sensors include fiber optic sensors disposed along the length of the fiber optic shape sensing device, wherein measuring one or more parameters includes measuring light spectra of surrounding tissues with the fiber optic shape sensing device, and wherein the distributed sensing device includes demarcations visible in an image and the step of assigning a location of an anatomical landmark includes assigning the location in the image using a demarcation.

14. The method as recited in claim 10, further comprising employing the location of the anatomical landmark to register the anatomical landmark with an image, wherein determining a transition region includes dynamically referencing positions of the distributed sensing device using the location of the anatomical landmark assigned at the transition region and at least one other reference point, and wherein assigning the location of an anatomical landmark using the transition region includes assigning the location based upon a location of a nearest sensor to the transition region.

15. A method for identifying an internal anatomical landmark, comprising:
inserting a distributed fiber optic sensing device into a volume of a body;
extending at least a portion of a length of the distributed sensing device beyond an area of interest such that the length of the distributed sensing device includes sensors located within the body on different sides of the point of interest;
measuring one or more parameters from surrounding tissue using the sensors located along the length of the distributed sensing device;
determining a transition region where a gradient point occurs between the sensors to associate the gradient point with one or more positions of the sensors along the length; and
assigning a location of an internal anatomical landmark to a sensor nearest to the gradient point.
